(19) United States
(12) Patent Application Publication
(10) Pub. No.: US 2024/0430605 A1
(43) Pub. Date: Dec. 26, 2024

(54) Microphone Natural Speech Capture Voice Dictation System and Method

(71) Applicant: BRAGI GmbH, München (DE)

(72) Inventor: Peter Vincent Boesen, München (DE)

(73) Assignee: BRAGI GmbH, München (DE)

(21) Appl. No.: 18/830,253

(22) Filed: Sep. 10, 2024

Related U.S. Application Data
(63) Continuation of application No. 17/938,822, filed on Oct. 7, 2022, now Pat. No. 12,088,985.
Application No. 17/938,822 is a continuation of application No. 17/159,695, filed on Jan. 27, 2021, now Pat. No. 11,496,827.
Application No. 17/159,695 is a continuation of application No. 15/946,100, filed on Apr. 5, 2018, now Pat. No. 10,904,653.
Application No. 15/946,100 is a continuation of application No. 15/383,809, filed on Dec. 19, 2016, now Pat. No. 9,980,033.
(60) Provisional application No. 62/270,419, filed on Dec. 21, 2015.

(51) Int. Cl. H04R 1/10; G06F 40/174; G10L 15/22; G10L 15/26; G16H 10/60; G16Z 99/00; H04R 1/08; H04R 5/033

(52) U.S. Cl. CPC H04R 1/1041; G06F 40/174; G10L 15/22; G10L 15/26; G16H 10/60; G16Z 99/00; H04R 1/08; H04R 1/1016; H04R 1/1083; H04R 5/0335; G10L 2015/227; H04R 2460/13

(57) ABSTRACT

A system for voice dictation includes an earpiece, the earpiece may include an earpiece housing sized to fit into an external auditory canal of a user and block the external auditory canal, a first microphone operatively connected to the earpiece housing and positioned to be isolated from ambient sound when the earpiece housing is fitted into the external auditory canal, a second microphone operatively connected to earpiece housing and positioned to sound external from the user, and a processor disposed within the earpiece housing and operatively connected to the first microphone and the second microphone. The system may further include a software application executing on a computing device which provides for receiving the first voice audio stream into a first position of a record and receiving the second voice audio stream into a second position of the record.

### PRIORITY STATEMENT

This application is a continuation of U.S. patent application Ser. No. 17/938,822 filed on Oct. 7, 2022 now U.S. Pat. No. 12,088,985, which is a continuation of U.S. patent application Ser. No. 17/159,695 filed on Jan. 27, 2021 now U.S. Pat. No. 11,496,827, which is a continuation of U.S. patent application Ser. No. 15/946,100 filed on Apr. 5, 2018 now U.S. Pat. No. 10,904,653, which is a continuation of U.S. patent application Ser. No. 15/383,809 filed on Dec. 19, 2016 now U.S. Pat. No. 9,980,033, which claims priority to U.S. Provisional Patent Application No. 62/270,419 filed on Dec. 21, 2015, all of which are titled Microphone Natural Speech Capture Voice Dictation System and Method, all of which are hereby incorporated by reference in their entireties.

### FIELD OF THE INVENTION

The present invention relates to wearable devices. More particularly, but not exclusively, the present invention relates to ear pieces.

### BACKGROUND

The patient medical record is the essential document of the medical profession which accurately and adequately captures the details of each patient encounter. Over the years, the requirements of the document have changed, as electronic medical records have added significant new levels of data required for processing. Such new burdens have significant impact on the health care providers, both personally and professionally. On a professional level, these new demands require protracted lengths of time to fulfill the demands of documentation. Additionally, these demands require health care professionals to spend an increasing segment of their time documenting the patient visit. This removes them from what they are trained to do: patient care. On a personal level, such increasing demands are the source of frustration, fatigue and increasing dissatisfaction. Therefore, what is needed is a new system that effectively captures critical data for the documentation process at the point of service.

### SUMMARY

Therefore, it is a primary object, feature, or advantage of the present invention to improve over the state of the art.

It is a further object, feature, or advantage of the present invention to provide for accurate accuracy in the voice capture of a user of a wearable device.

It is a still further object, feature, or advantage of the present invention to markedly improve data capture from a wearable user due to isolation of the bone microphone.

Another object, feature, or advantage is to acquire patient voice signals in real time, using an external facing microphone to detect patient voice inputs.

Yet another object, feature, or advantage is to allow for instantaneous voice to text conversion.

A further object, feature, or advantage is to allow for capture of a voice snippet at a position within a document.

A still further object, feature, or advantage to allow for editing and correction of incorrect segments of the voice to text conversion.

Another object, feature, or advantage is to allow for standard edits to other non-voice sections of a document.

Yet another object, feature, or advantage is to allow for insertion of voice to text snippets at the direction of the primary user, in this case the health care provider.

A further object, feature, or advantage is to allow for the capture of the patient encounter at the point of service, greatly improving accuracy while simultaneously saving time and money.

A still further object, feature, or advantage is to reduce healthcare administrative costs.

Yet another object, feature, or advantage is to collect contextual sensor data at an earpiece.

A further object, feature, or advantage is to create a record and/or interpret nonverbal information as a part of a transcript of a communication.

One or more of these and/or other objects, features, or advantages of the present invention will become apparent from the specification and claims that follow. No single embodiment need provide every object, feature, or advantage. Different embodiments may have different objects, features, or advantages. Therefore, the present invention is not to be limited to or by any objects, features, or advantages stated herein.

A new and novel way of capturing patient information at the point of service is provided. Such a system may be able to distinguish between a physician voice and a patient's voice. The system may use a combination of microphones. The first microphone may be in the external auditory canal of the healthcare provider. It may be optimized to pick up the “Self-voice” of the healthcare provider. This has the distinct advantage of being acoustically isolated in the external canal of the healthcare provider while providing the optimal environment for capturing the “self-voice” of the primary user. The external microphone may be optimized to pick up the vocal sounds from the patient in the room. In doing so, the healthcare user's microphone would be able to discern the difference between the two voices based upon microphone inputs. This allows the optimized speech engine to segregate the two voice inputs. Such inputs can then be directly inputted into the patient record, stored in the selected position within the record as a voice file, or both. In this fashion, the system may provide the ultimate in flexibility to rapidly and accurate capture the conversation between a healthcare worker and patient, convert to text while at the same time allowing for review or modification as needed. Such editing capability allows the user to have the ability to edit all aspects of the document before their electronic signature.

According to one aspect, a system for voice dictation is provided. The system includes an earpiece. The earpiece includes an earpiece housing sized to fit into an external auditory canal of a user and block the external auditory canal, a first microphone operatively connected to the earpiece housing and positioned to be isolated from ambient sound when the earpiece housing is fitted into the external auditory canal, a second microphone operatively connected to earpiece housing and positioned to sound external from the user, and a processor disposed within the earpiece housing and operatively connected to the first microphone and the second microphone. The processor is adapted to capture a first voice audio stream using at least the first microphone, the first voice audio stream associated with the user, and a second voice audio stream using at least the second microphone, the second voice audio stream associated with a person other than the user. The system may also include a software application executing on a computing device which provides for receiving the first voice audio stream into a first position of a record and receiving the second voice audio stream into a second position of the record.

According to another aspect, a method for voice dictation is provided. The method includes providing an earpiece, the earpiece having an earpiece housing sized to fit into an external auditory canal of a user and block the external auditory canal, a first microphone operatively connected to the earpiece housing and positioned to be isolated from ambient sound when the earpiece housing is fitted into the external auditory canal, a second microphone operatively connected to earpiece housing and positioned to sound external from the user; and a processor disposed within the earpiece housing and operatively connected to the first microphone and the second microphone. The processor is adapted to capture a first voice audio stream using at least the first microphone, the first voice audio stream associated with the user, and a second voice audio stream using at least the second microphone, the second voice audio stream associated with a person other than the user. The method further includes capturing a first voice audio stream using at least the first microphone, the first voice audio stream associated with the user, storing the first voice audio stream on a machine readable storage medium, converting the first voice audio stream to text, placing the text within a first form field in a software application, and providing access to the first voice audio stream through the software application.

### BRIEF DESCRIPTION OF THE DRAWINGS

FIG. 1 illustrates one example of a system.

FIG. 2 illustrates a set of earpieces in greater detail.

FIG. 3 illustrates a block diagram of one of the earpieces.

FIG. 4 illustrates one example of a screen display from a software application.

FIG. 5 illustrates one example of a screen display from a word processor.

FIG. 6 illustrates one example of a screen display from a medical record application.

FIG. 7 illustrates one example of a screen display for a software application where contextual feedback is sensed by the earpiece and received into the software application.

### DETAILED DESCRIPTION

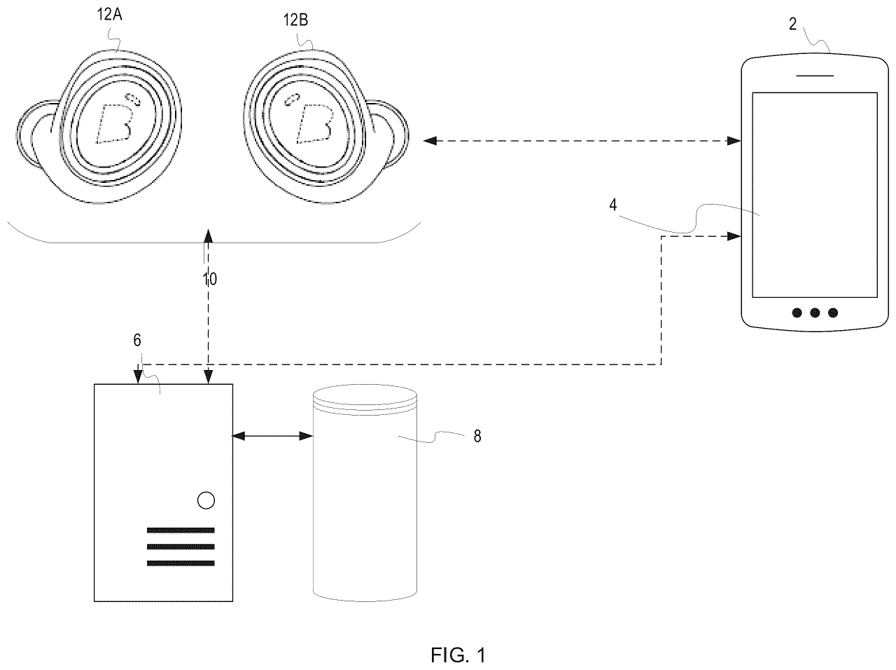

FIG. 1 illustrates one example of a system. As shown in FIG. 1 there are one or more earpieces 10 such as a left earpiece 12A and a right earpiece 12B. Although multiple earpieces are shown, only a single earpiece may be used. The earpieces 12A, 12B may be in operative communication with a computing device 2. The computing device 2 may be a computer, a mobile device such as a phone or tablet, or other type of computing device. There may be a display 4 associated with the computing device 2. A server 6 is also shown. The server 6 is in operative communication with a data store 8 such as a database. The server 6 may be a cloud-based server, a physical server, a virtual server executing on a hardware platform, or other type of server.

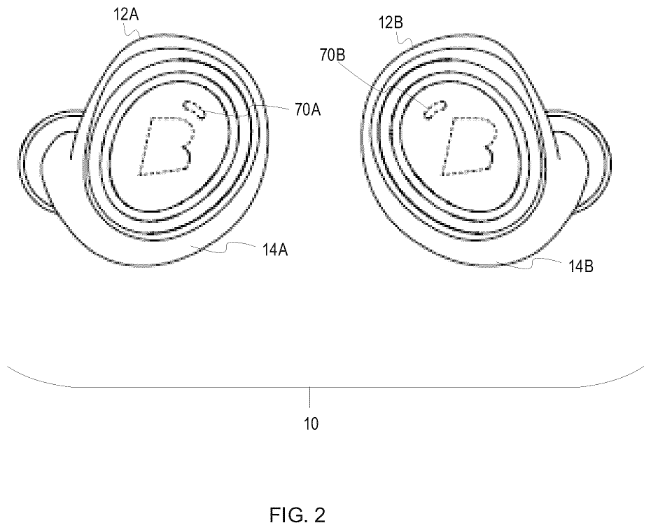

FIG. 2 illustrates a set of earpieces 10 in greater detail. A left earpiece 12A is housed within an earpiece housing 14A. The left earpiece 12A includes an outward facing microphone 70A. The right earpiece 12B is housed within an earpiece housing 14B. The right earpiece 12B includes an outward facing microphone 70B. The earpieces may be the earpieces which are commercially available from Bragi GmbH such as THE DASH.

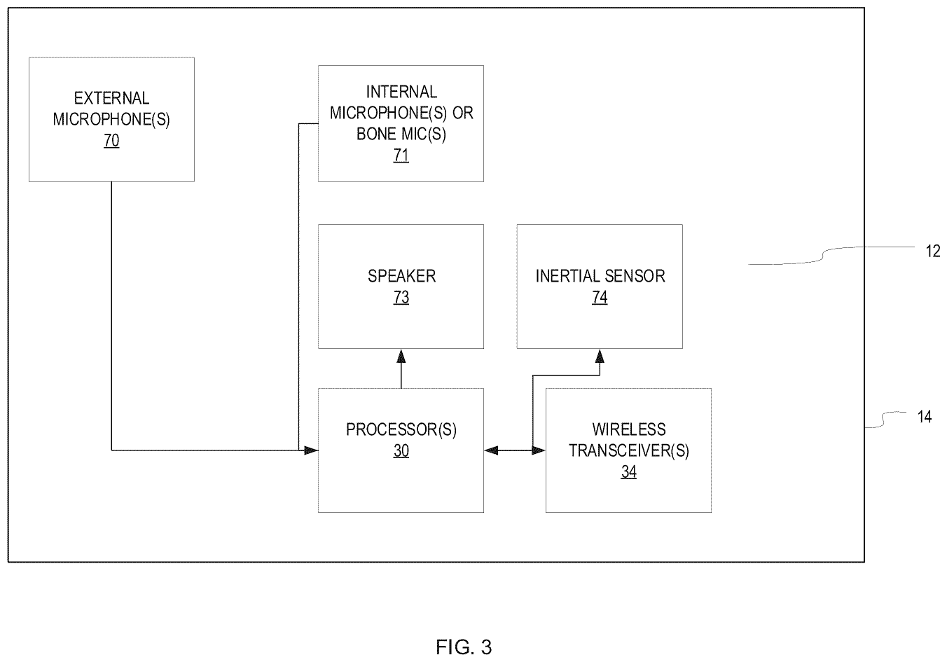

FIG. 3 illustrates a block diagram of one of the earpieces 12. The earpiece 12 has an earpiece housing 14. Disposed within the earpiece housing is at least one processor 30. The processor 30 is operatively connected to at least one wireless transceiver 34 which may include a radio transceiver capable of communications using Bluetooth, BLE, Wi-Fi, or other type of radio communication. One or more external microphones 70 and one or more internal microphones 71 are also operatively connected to the processor 30. In addition, a speaker 73 is operatively connected to the processor 30. Note that the external microphone(s) 70 may be positioned to detect or capture voice streams associated with one or more speakers other than the person wearing the earpiece (the user). The one or more internal microphones 71 may be, for example, positioned at or near the external auditory canal or mastoid bone of the user and may provide for picking-up bone vibrations or are otherwise configured to pick up frequency ranges associated with the person wearing the earpiece. In addition, there may be one or more inertial sensors 74 present in the earpiece 12. The inertial sensor may include a gyroscope, accelerometer, or magnetometer. For example, the inertial sensor 74 may be a 9-axis accelerometer which includes a 3-axis gyroscope, a 3-axis accelerometer, and a 3-axis magnetometer.

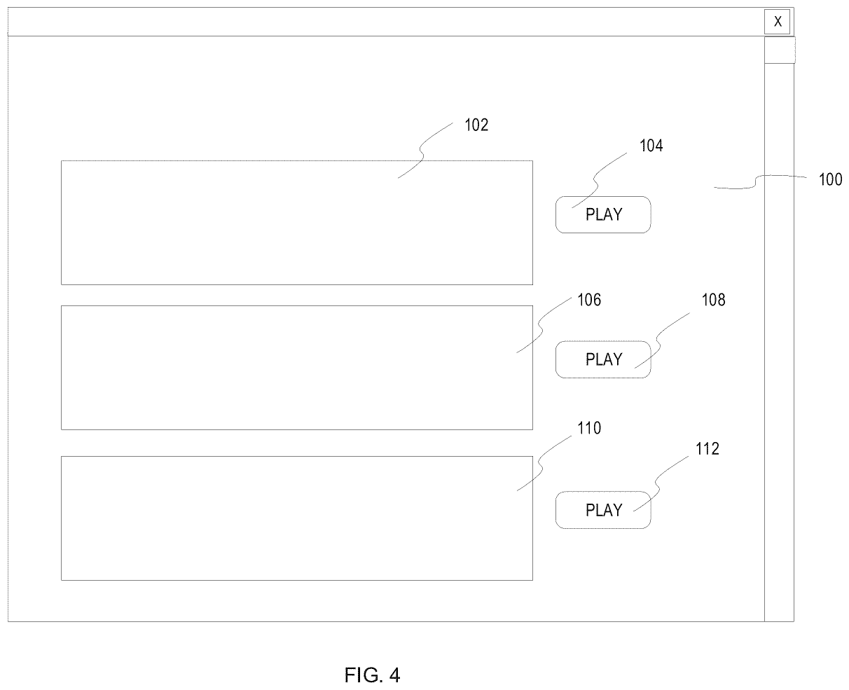

FIG. 4 illustrates one example of a software application which includes a screen display 100. Various form fields 102, 106, 110 are shown. In one embodiment, each time a different speaker (e.g. person) speaks, the software application moves to the next form field. Each form field is populated with text acquired from conversion of voice information to text information. In addition, to this representation of the translated text, the underlying voice stream or voice recording may be played by selecting the corresponding play button 104, 108, 112. Thus, information from multiple individuals may be collected. It is of further note, where the earpiece includes separate microphones for external users and the user of the ear pieces, that separate voice streams may be captured even when the user of the earpieces and another individual are talking at the same time. It is to be further contemplated that there may be more one other individual who is speaking who is within the environment of the user.

Capturing and storing the voice streams or voice snippets and associating these voice streams or voice snippets with the text may provide additional advantages. There is a complete record so that if need be the text information may be correctly later or date if it does not accurately match the voice snippet.

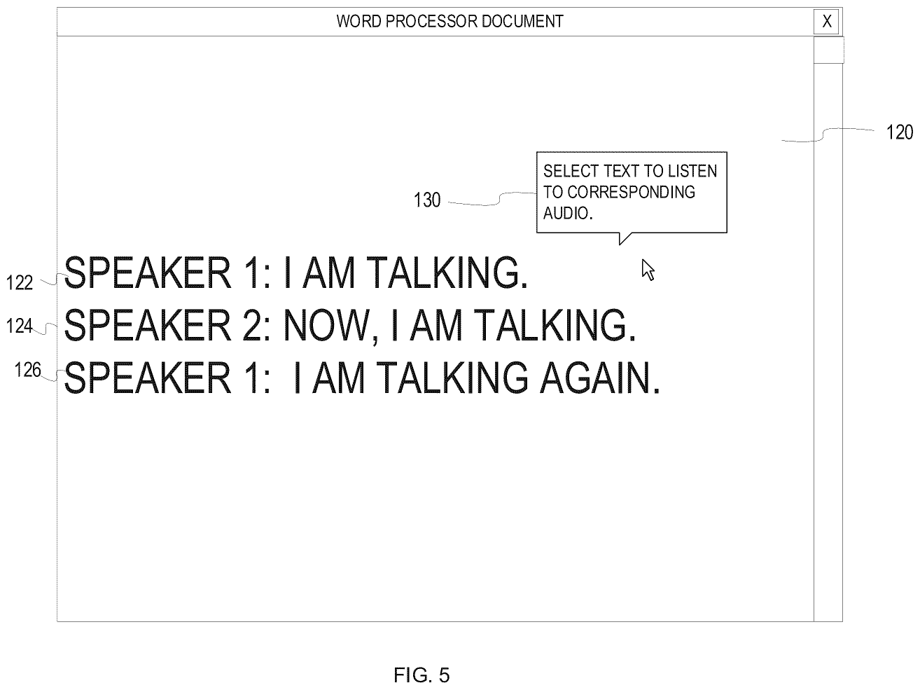

FIG. 5 illustrates another example of a software application that may be used. As shown in FIG. 5, there is a screen display 120 which may be associated with a word processor document. The word processor may be a word processor such as Microsoft Word, the Microsoft Office Online version of Microsoft Word, WordPerfect, TextMaker, Pages from Apple, Corel Write, Google Docs, or any other word processor. The word processor software may execute on a local machine or on a remote machine such as available through cloud or web access. Functionality may be built-into the word processor or may be provided as an add-in, as a connected application, or otherwise.

As shown in FIG. 5, a transcript may be created which includes text from multiple different speakers. As shown, each speaker may be identified such as “Speaker 1”, “Speaker 2.” Alternatively, each speaker may be given a name. Also, instead of or in addition to identifying speakers in this fashion, text associated with different speakers may be presented in different colors of text, different fonts, or different styles. As shown in FIG. 5, an icon may be shown associated with a mouse or other control device. The mouse or other control device may be used to select a portion of the text. When that portion of the text is selected, the corresponding audio may be played. Thus, if there appears to be a transcription error in the text, a user may confirm whether there was a transcription error or not. Alternatively, a portion of text may be otherwise selected such as by selecting an icon associated with that portion of the text. Thus, as shown a first speaker may make a first statement 122, a second speaker may make a second statement 124, and the first speaker may make a third statement 126. A tooltip 130 is shown indicating that a user can choose to select text to listen to corresponding audio.

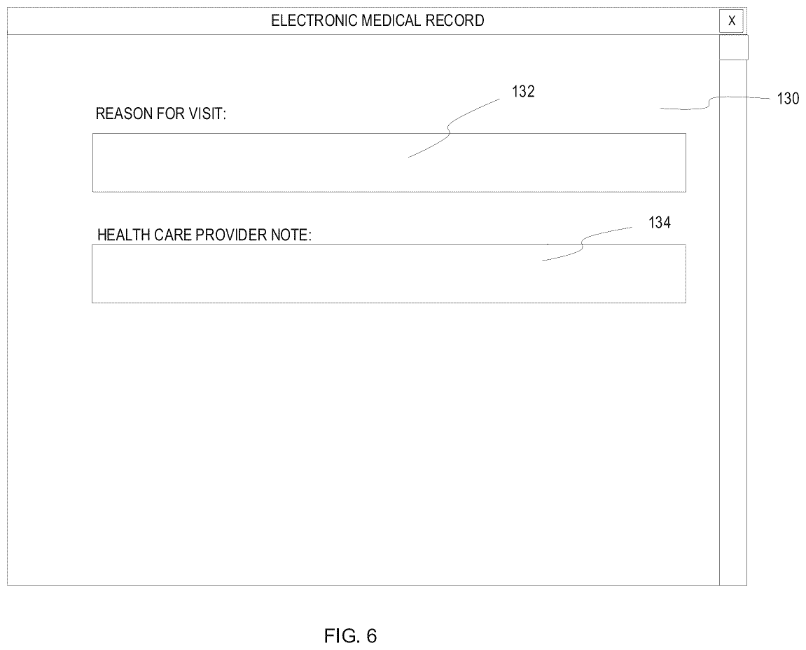

FIG. 6 illustrates another example of a software application. FIG. 6 illustrates a screen display 130 associated with an electronic medical record (EMR), electronic health record (EHR), electronic patient record (EPR), or other type of medical record. In the context of a medical record, it is contemplated that information entered into a medical record may come from words dictated by a health care provider or from information obtained orally from a patient. The earpiece described herein may be used to collect audio from both the health care provider (such as by using a bone conduction microphone) and from the patient (such as by using an external facing microphone). For example, as shown in FIG. 6 voice information associated with the reason for the visit, as spoken by a patient, may be input as text into form field 132 and a recording of the audio may be associated with this form field. In addition, voice information, as spoken by the health care provider, may be input as text into form field 134 and a recording of the audio may be associated with this form field. Although given as an example in the context of the medical field, any number of other situations may be appropriate where a transcript of an encounter is desired.

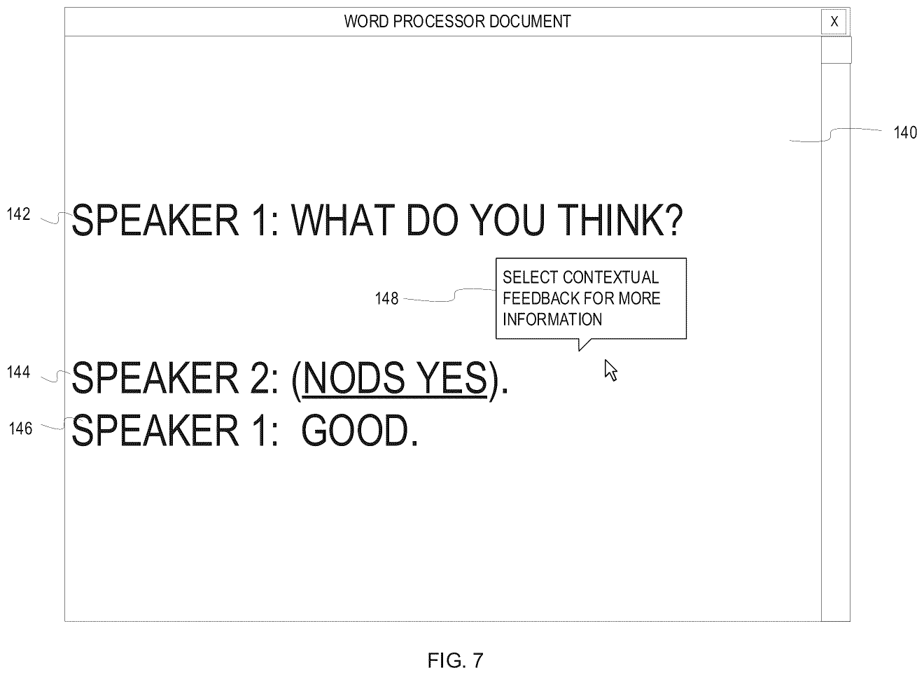

FIG. 7 illustrates another example of a screen display 140. As shown in FIG. 7, a transcript may be created which includes text from multiple different speakers. As shown, each speaker may be identified such as “Speaker 1”, “Speaker 2.” Alternatively, each speaker may be given a name. In addition to a transcript of text, the ear piece may include other information sensed by the ear piece. For example, where the ear piece includes an inertial sensor, information associated with the inertial sensor or a characterization of information associated with the inertial sensor may be included. In this example, “Speaker 2” is wearing the earpiece. The statement 142 made by Speaker 1 may be detected with an externally facing microphone of an earpiece worn by Speaker 2. In response to statement 142, Speaker 2 may nod their head in agreement. This gesture or movement associated with the head nod may be detected with one or more inertial sensors of the earpiece. This head movement or a record of it may then be incorporated into the transcript. The record of the head movement 146 may be shown in a manner distinct from the voice transcript such as using different colors, fonts, or styles, such as underlining, including in parentheses, or otherwise. In addition, additional information may be obtained by selecting the inserted text indicating that the nod occurred. The additional information may be in the form of raw sensor data, or other characterization of the nod or other sensor data. Examples of different characterizations may include the degree of the head nod or characterization of how pronounced the head nod is. The characterizations may be quantitative or qualitative. A tooltip 148 may be shown indicating that a user may select the contextual feedback to access this additional information. In addition to head nods, other gestures may also be detected. This may include a head shaking movement, such as may be associated with a “NO.” Although gestures as detected with inertial sensors are one type of movement which may detected to provide contextual feedback, it is contemplated that other types of contextual feedback may be used such as may be detected through physiological monitoring or otherwise. Other types of sensors may also include image sensors. Where image sensors are used, the image sensors may be used to detect information from either the individual wearing the earpiece or other wearable device or from others. Thus, records may be created for nonverbal information as a part of a transcript of a communication or as input into different fields within a document or software application.

In another embodiment, a person is using the earpieces on a phone call and the voice of the person on the other side of the call is captured and transcribed as opposed to capturing voice of a person through one or more microphones on the ear piece. In yet another embodiment, a conversation may occur either in person or over a communication network with two or more individuals with at least two of the individuals wearing earpieces so that contextual information from more than one person may be captured as a part of the conversation.

Therefore, methods and systems for voice dictation using one or more earpieces have been shown and described. Although specific embodiments are shown here, it is contemplated that any number of options, variations, and alternatives may also be used. The present invention is not to be limited unduly to specifically what is shown and described herein.

1. A system for voice dictation, the system comprising:
an earpiece, the earpiece comprising:
an earpiece housing;
a first microphone operatively connected to the earpiece housing and positioned to detect a voice of a user;
a second microphone operatively connected to earpiece housing and positioned to detect a sound external from the user;
a processor disposed within the earpiece housing and operatively connected to the first microphone and the second microphone, wherein the processor is adapted to capture a first voice audio stream using at least the first microphone, the first voice audio stream associated with the user, and a second voice audio stream using at least the second microphone, the second voice audio stream associated with a person other than the user;
an inertial sensor comprising an accelerometer and a gyroscope, the inertial sensor disposed within the earpiece housing and operatively connected to the processor; and
a software application executing on a computing device in wireless communication with the earpiece which provides generating a screen display showing a record having a first field at a first position, a second field at a second position, and a third field at a third position, wherein the software application further providing for inputting the first voice audio stream into the first field at the first position of the record on the screen display, the second voice audio stream into the second field at the second position of the record on the screen display, and contextual data based on head movement from the user into the third position.

2. The system of claim 1 wherein the record is a medical record, the user is a health care provider and the person other than the user is a patient.

3. The system of claim 2 wherein the software application provides for converting the first voice audio stream into a first audio file, storing the first audio file, converting the first voice audio stream into first text and placing both the first text and a first link to the first audio file at the first position of the record.

4. The system of claim 3 wherein the software application provides for converting the second voice audio stream into a second audio file, storing the second audio file, converting the second voice audio stream into second text and placing both the second text and a second link to the second audio file at the second position of the record.

5. The system of claim 1, wherein the processor is configured to interpret input from an inertial sensor as the head movement.

6. The system of claim 5 wherein the processor is configured to interpret the head movement as indicative of a yes.

7. The system of claim 5 wherein the processor is configured to interpret the head movement as indicative of a no.

8. A method for voice dictation, the method comprising:
providing a computing system, worn on a head of a user, the computing system comprising:
a first microphone positioned to detect a voice of the user;
a second microphone positioned to receive a sound external from the user;
a processor disposed operatively connected to the first microphone and the second microphone; and
an inertial sensor positioned on the user in operative communication with the processor;
capturing a first voice audio stream using at least the first microphone, the first voice audio stream associated with the user;
capturing inertial sensor data with the inertial sensor and interpreting the inertial sensor data into contextual data;
storing the first voice audio stream on a machine readable storage medium;
converting the first voice audio stream to first text;
placing the first text within a user interface of the screen display; and
providing user controls on the screen display to provide access to the first voice audio stream and the contextual data through the software application.

9. The method of claim 8 wherein the first microphone is a bone microphone.

10. The method of claim 8 further comprising:
capturing a second voice audio stream using the second microphone, the second voice audio stream associated with a person other than the user;
storing the second voice audio stream on a machine readable storage medium;
converting the second voice audio stream to second text;
placing the second text of the second voice audio stream within the user interface of the screen display; and
providing user controls on the screen display to provide access to the second voice audio stream through the software application.

11. The method of claim 10 wherein the software application is a medical records software application.

12. The method of claim 11 wherein the user is a health care provider and wherein the person other than the user is a patient of the health care provider.

13. The method of claim 12 wherein the voice dictation is performed during a patient encounter to document the patient encounter.

14. The method of claim 10 further comprising receiving a correction of the first text from the user and updating the first text with the correction.

15. The method of claim 8 further comprising capturing a second voice audio stream at a wireless transceiver operatively connected to the computing system.

16. The method of claim 15 further comprising converting the second voice audio stream to second text.

17. The method of claim 8 further comprising capturing sensor data with the computing system and interpreting the sensor data into text data and placing the text data into the user interface of the screen display within the software application.

18. The system of claim 17, wherein the software application provides for indicating the occurrence of the head movement by the user.

19. The system of claim 18 wherein the head movement is indicative of a yes.

20. The system of claim 18 wherein the head movement is indicative of a no.
